(19) United States
(12) Patent Application Publication
(10) Pub. No.: US 2013/0156868 A1
(43) Pub. Date: Jun. 20, 2013

(54) NASAL SPRAY

(71) Applicant: Detlef Schierstedt, St. Augustin (DE)

(72) Inventor: Detlef Schierstedt, St. Augustin (DE)

(73) Assignee: Krewel Mueselbach GmbH, Eitorf (DE)

(21) Appl. No.: 13/817,539

(22) PCT Filed: Sep. 1, 2011
(86) PCT No.: PCT/EP2011/065132

§ 371 Date: Feb. 19, 2013

(30) Foreign Application Priority Data
Sep. 7, 2010 (DE) 202010012255.4

(51) Int. Cl. A61K 36/534; A61K 31/164; A61K 33/14; A61K 31/045

(52) U.S. Cl. CPC A61K 36/534; A61K 31/045; A61K 31/164; A61K 33/14
USPC 424/680; 514/729; 424/745; 514/625; 514/692

(57) ABSTRACT

The invention relates to a medicinal product which is free from sympathomimetics, has an advantageous effect on the nasal mucosa in case of common colds, hay fever, dry nose and sympathomimetic dependencies, and is provided in the form of a nasal spray, a nasal douche or nasal drops.

The invention relates to a sympathomimetic-free medicinal product having an advantageous effect on the nasal mucosa in common cold, hay fever or other rhinitides, dry nose and/or sympathomimetic dependences, in the form of a nasal spray, rinse, or nose drops.

In common cold (rhinitis catarrhalis), hay fever, other rhinitides or dry nose, the nasal mucosa is subject to swelling. To date, it has been usual to use sympathomimetics locally in the form of nose drops or nasal sprays for treatment in order to achieve reduction of mucosal swelling. However, sympathomimetics have numerous undesirable side effects and may lead to palpitations and respiratory disorders because of absorption. In addition, they cause the nasal mucosa to dry out, and when applied for extended periods of time, permanent damage to the nasal mucosal epithelium may occur. Such a sympathomimetic dependence is known as rhinitis medicamentosa (Apotheker-Journal 12, 30-34 (1985), Otto Hoffmanns Verlag, Munich).

EP 0 216 917 B1 relates to a therapeutic preparation for nasal administration that contains a thickening agent, among others. For example, methylcellulose is mentioned as said thickening agent. U.S. Pat. No. 5,843,881 A relates to a spray composition. In particular, the composition contains alcohol, a polymer and an alcohol-masking perfume additive. The compositions are applied to the skin, the hair or the mucosa.

In recent years, sympathomimetic-free nasal sprays based on sea salt and/or common salt have become established. These do not have an acute pharmacological effect (medicinal product) on the nasal mucosa, but are able to soothe the mucosa and thus cause an alleviation of complaints, especially after several applications, by moistening and cleaning the mucosa.

Since the dwelling time of the solution is short, the solution must be applied very frequently, and the alleviation is often insufficient when the complaints are severe.

Therefore, it is the object of the present invention to provide a medicinal product that has a stronger soothing effect on the nasal mucosa as compared to known products. However, it should not cause dependences, as is the case with sympathomimetic-containing products.

Surprisingly, it has been found that menthol, mint oil and/or camphor together with a humectant have a stronger clearing effect on the nasal mucosa than that of the usual sprays containing common salt and/or sea salt. A particular embodiment additionally contains panthenol, especially dexpanthenol.

Therefore, a first embodiment of the invention includes a sympathomimetic-free medicinal product for soothing the nasal mucosa, characterized by comprising menthol, mint oil and panthenol, especially dexpanthenol, as well as at least one humectant comprising water-soluble or water-dispersible natural or synthetic polymers that form gels or viscous solutions in aqueous systems, polyhydric alcohols, mono-, di- and/or polysaccharides. Polymers are preferably used. Suitable polymers include, for example, collagen derivatives (animal and plant derived), polyalkylene glycols, especially polyethylene glycols, polyglycerols, alginates, carrageenan, pectins, tragacanth gum, gums, especially gum arabic, cellulose derivatives, especially cellulose ethers and/or cellulose esters, polyvinyl alcohols, polyvinyl pyrrolidones and derivatives thereof, and/or dextran.

Particularly preferred cellulose derivatives within the meaning of the present invention include hydroxyethylcellulose and/or methylhydroxypropylcellulose.

The medicinal product according to the invention may further contain camphor.

The combination of humectant and panthenol together with menthol and mint oil and optionally camphor causes humidification of the nasal mucosa. In addition, the nasal mucosa comes to be soothed. Menthol and mint oil and optional camphor have a cooling effect on the nasal mucosa. In combination with the humectant, the nasal mucosa comes to be soothed. Surprisingly, it has been found that these two physical effects complement each other perfectly in a nasal spray according to the invention, and have an improved effect as compared to nasal sprays containing sea salt and/or common salt.

“Soothing the nasal mucosa” within the meaning of the present invention means that the nasal mucosa is kept humid. The feeling of dryness that occurs when sympathomimetic-containing nasal sprays are used, does not occur with the nasal spray according to the invention. In addition, “soothing” also includes a lesser swelling or even reduction of swelling of the irritated nasal mucosa.

When used on a regular basis, the preparation according to the invention is an alternative to sympathomimetic-containing nasal sprays. This may also be beneficial to those persons who depend on sympathomimetic-containing nasal spray.

Several subjects, who normally depended on the use of sympathomimetic-containing sprays when affected by a common cold, received the product according to the invention in the form of a spray over a period of one week. Two subjects were able to completely dispense with sympathomimetic-containing sprays during the infection period of one week. Four subjects reduced the use of sympathomimetic-containing sprays to half while they employed the product according to the invention.

Five other subjects who were dependent on sympathomimetic-containing nasal sprays alternatively used the spray according to the invention. To be able to breathe deeply in the nighttime, the subjects previously used sympathomimetic-containing sprays before going to sleep, which were entirely or partially replaced by the spray according to the invention. After two weeks, one of the five subjects could completely dispense with the use of sympathomimetic-containing nasal sprays. The others could reduce the use of the sympathomimetic-containing sprays to about half.

According to the invention, the medicinal product may contain at least one humectant in an amount of from 0.1 to 5% by weight, especially in an amount of from 0.5 to 2% by weight.

If the amount of the at least one humectant is too low, the formation of a sustainable film is prevented, while if the amount of the at least one humectant is too high, the viscosity of the medicinal products is undesirably increased.

In another embodiment, the medicinal product according to the invention contains mint oil and menthol and optionally camphor up to their saturation limit. With higher concentrations, a homogeneous phase in the medicinal product according to the invention cannot be ensured. Thus, the concentration of mint oil and menthol and optionally camphor would vary widely for each application. In addition, the nasal mucosa would come to be inhomogeneously wetted during the application, not only with mint oil, menthol and/or camphor. A homogeneous application of optional panthenol and humectant cannot be ensured either.

A medicinal product according to the invention preferably contains more than 0.002 g, especially more than 0.003 g, of mint oil and menthol. If camphor is present in the medicinal product, the medicinal product preferably contains more than 0.002 g, especially more than 0.003 g, of camphor. With lower contents, a sufficient soothing effect on the nasal mucosa is not ensured.

Another preferred embodiment of the present invention is characterized in that the medicinal product further additionally contains common salt and/or sea salt. Instead of or in combination with it, other tonicizing additives, such as physiological salts, buffers, or ionic or non-ionic physiologically tolerable substances, may serve as the basis. The use of common salt or sea salt is widespread in the prior art. Corresponding agents may contain natural sea water with trace elements and minerals.

By means of pH control agents, it is possible to adjust the pH value of the medicinal products according to the invention to a physiologically tolerable pH, not below 6.5 if possible. Particularly preferred pH control agents within the meaning of the present invention include sodium hydroxide, sodium phosphate, sodium citrate and other physiologically tolerable buffer systems, for example, phosphate or citrate buffer. The above mentioned limit corresponds to the pH value usual for a nasal spray, which is determined by the mucosa compatibility.

The tonicity of the medicinal products according to the invention is usually adjusted towards a slight hypertonicity (more preferably 400 mosmol). Particularly preferred tonicizing agents include, in particular, glucose, sorbitol, mannitol and/or xylitol. With a hypotonic solution (less than 290 mosmol), there is a risk that the cells of the nasal mucosa are damaged. Strongly hypertonic solutions are found unpleasant.

The galenic formulation of the medicinal products according to the invention can be effected according to the galenic methods and rules that are generally usual for the preparation of aqueous nose drops (H. Sucker, P. Fuchs and P. Speiser: Pharmazeutische Technologie, Thieme Verlag, Stuttgart (1978)). More preferably according to the present invention, the medicinal products are prepared in the form of an aqueous solution.

In another embodiment, the medicinal product further comprises essential oils. Thyme oil, eucalyptus oil and/or sage oil are preferred according to the present invention. These provide for a pleasant smell of the medicinal product.

A sympathomimetic-free medicinal product according to the invention is preferably free of preservatives. Preservatives would reduce the decongestant effect on the nasal mucosa. Moreover, they frequently again provoke irritation of the nasal mucosa and damage the cilia of the nose.

According to the invention, in particular, in another embodiment, the medicinal product is in the form of an aqueous solution. The essential oils can be added to such an aqueous solution up to their maximum solubility.

Another embodiment relates to the use of the medicinal product according to the invention. It is particularly preferred to use it for irritations of the nasal mucosa caused by a cold or by hay fever. They may also be caused by a dry nose as a result of dry air from heating systems, among others. Also, irritations of the nasal mucosa because of sympathomimetic dependence can be alleviated by the medicinal product according to the invention. Preferably, the medicinal product according to the present invention is used in the form of drops, sprays and/or rinses.

### EXAMPLES

### Example 1

| | | |
|---|---|---|
| Methocel ® E5 | 2.0 | g |
| Dexpanthenol | 1.0 | g |
| Mint oil | 0.08 | g |
| Menthol | 0.016 | g |
| Sea salt | 0.9 | g |

### Example 2

| | | |
|---|---|---|
| Methocel ® E5 | 2.0 | g |
| Dexpanthenol | 1.0 | g |
| Mint oil | 0.08 | g |
| Menthol | 0.016 | g |
| Sea salt | 0.9 | g |
| Thyme oil | 0.02 | g |
| Eucalyptus oil | 0.01 | g |

### Comparative Example 1

| | | | |
|---|---|---|---|
| Thyme oil | | 0.02 | g |
| Eucalyptus oil | | 0.01 | g |
| Sea salt | | 1.0 | g |
| | ad | 100 | ml |

1. A sympathomimetic-free medicinal product in the form of drops, spays, and/or rinses for use for preparing an agent for treating irritations of the nasal mucosa by a cold, by hay fever, a dry nose and/or sympathomimetic dependence, characterized by comprising menthol, mint oil and/or camphor as well as at least one humectant comprising water-soluble or water-dispersible natural or synthetic polymers that form gels or viscous solutions in aqueous systems, polyhydric alcohols, mono-, di- and/or polysaccharides.

2. The sympathomimetic-free medicinal product according to claim 1, characterized in that said polymers are selected from collagen derivatives, polyalkylene glycols, especially polyethylene glycols, polyglycerols, alginates, carrageenan, pectins, gums, cellulose derivatives, polyvinyl alcohols, polyvinyl pyrrolidones and derivatives thereof, and/or dextran.

3. The sympathomimetic-free medicinal product according to claim 1, characterized by further containing panthenol.

4. The sympathomimetic-free medicinal product according to claim 1, characterized by containing said humectant in an amount of from 0.1 to 5% by weight.

5. The sympathomimetic-free medicinal product according to claim 1, characterized by further additionally containing common salt and/or sea salt.

6. The sympathomimetic-free medicinal product according to claim 1, characterized by additionally containing essential oils.

7. The sympathomimetic-free medicinal product according to claim 1, characterized by further containing pH control agents and/or tonicizing agents.

8. The sympathomimetic-free medicinal product according to claim 1 in the form of an aqueous solution.

9. Use of a sympathomimetic-free medicinal product according to claim 1 for preparing an agent for treating irritations of the nasal mucosa caused by a cold, by hay fever, a dry nose and/or sympathomimetic dependence.

10. The use according to claim 9 in the form of drops, sprays and/or rinses.

11. The sympathomimetic-free medicinal product according to claim 4, wherein said humectants is in an amount of 0.5-2% by weight based on total weight of the product.

12. The sympathomimetic-free medicinal product according to claim 6, wherein said essential oil includes thyme oil, eucalyptus oil, and/or sage oil.

13. The sympathomimetic-free medicinal product according to claim 7, wherein said tonicizing agent includes glucose, sorbitol, mannitol and/or xylitol.

14. The sympathomimetic-free medicinal product according to claim 2, wherein said gums include tragacanth gum and/or gum Arabic.

15. The sympathomimetic-free medicinal product according to claim 2, wherein said cellulose derivatives include cellulose ethers and/or cellulose esters.
